(19) United States
(12) Patent Application Publication
(10) Pub. No.: US 2020/0024580 A1
(43) Pub. Date: Jan. 23, 2020

(54) APPLICATION OF UTX GENE IN PREPARATION OF DRUGS FOR PREVENTING OR TREATING LIPID DISEASES

(71) Applicant: Nanjing Maternity and Child Health Care Hospital, Nanjing (CN)

(72) Inventors: Chenbo JI, Nanjing (CN); Ling CHEN, Nanjing (CN); Xirong GUO, Nanjing (CN); Hong ZHONG, Nanjing (CN)

(21) Appl. No.: 16/554,589

(22) Filed: Aug. 28, 2019

(30) Foreign Application Priority Data
Mar. 28, 2019 (CN) 2019102480171

(51) Int. Cl. C12N 9/02; C12N 15/52; C12N 15/86

(52) U.S. Cl. CPC C12N 9/0071; C12N 2015/8518; C12N 15/86; C12N 15/52

(57) ABSTRACT

An application of a UTX gene in preparation of drugs for preventing or treating lipid diseases. The invention further discloses a method for knocking out a UTX gene from a mouse liver. The invention further discloses a UTX overexpression adenovirus as well as a preparation method and an application thereof. The invention further discloses a method for upregulating UTX expression in a mouse liver. The invention further discloses therapeutic action of UTX overexpression on HFD induced hyperlipidemia and NAFLD. The invention provides an available laboratory basis for preparing lipid-lowering drugs, so that the UTX can be used for preparing drugs affecting the lipid, and a new research method is provided for researching the occurrence and development of dyslipidemia.

This application claims priority to Chinese Patent Application Ser. No. CN2019102480171 filed on 28 Mar. 2019.

### TECHNICAL FIELD

The present invention relates to the field of genetic engineering, and relates to an application of a UTX gene in preparation of drugs for preventing or treating lipid diseases.

### BACKGROUND

Histone methylation plays an important role in regulating metabolic diseases such as hyperlipidemia, obesity and non-alcoholic fatty liver disease (NAFLD). UTX (ubiquitously transcribed tetratricopeptide repeat on chromosome X) gene located on the X chromosome, is highly expressed in the liver, spleen, and brain tissues. As a histone demethylase that removes the di- and tri-methyl groups from histone H3K27 and activates gene expression, plays important roles in numerous biological and pathological processes. Previous study reported that UTX was upregulated in the renal mesangial and tubular cells of diabetic kidney disease and diabetic mice. In addition, the expression of hepatic gluconeogenesis gene PEPCK was significantly reduced when UTX knocked down in HepG2, indicating that UTX was involved in glucose metabolism in the body. Importantly, mice lacking UTX in adipocytes showed significantly decreased triacylglycerol synthesis activity, further revealing that UTX gene was closely related to lipid metabolism. However, the function of UTX in hyperlipidemia has not been reported.

Hyperlipidemia refers to high lipid level, which is caused by abnormal metabolism or transportation of cholesterol and triglyceride in blood. Studies have shown that the hyperlipidemia, as a high-risk factor for coronary heart disease, stroke, fatty liver, hyperuricemia and other diseases, is increased sharply and has a trend of younger age. “China's Cardiovascular Disease Report in 2010” showed that 18.6% of the population over 18 years old had dyslipidemia, and the number of patients had reached 200 million. According to statistics, 30 million people around the world die of related diseases caused by hyperlipidemia every year. It is known that the hyperlipidemia is the result of lipid metabolism disorder under the interaction of many factors such as heredity, environment and living behavior, and the pathogenesis is complex. Therefore, it is very important to carry out etiological research of hyperlipidemia, which will provide an important theoretical basis for clarifying the pathogenesis of hyperlipidemia. However, there are no reports on the function of the UTX in the hyperlipidemia.

In the previous research work, after adopting a genetic engineering technology to specifically knock out a UTX gene from a mouse liver tissue, the inventor found that the liver-specific UTX knockout mouse showed obvious hypercholesterolemia and hypertriglyceridemia, which suggested that it might be related to the occurrence and development of hyperlipidemia.

### SUMMARY

Object of the invention: the technical problem to be solved by the present invention is to provide an application of a UTX gene in preparation of drugs for preventing or treating lipid diseases.

The technical problem to be solved by the present invention is to provide an application of a UTX gene in preparation of lipid-lowering drugs.

The technical problem to be further solved by the present invention is to provide a method for knocking out a UTX gene from a mouse liver.

The technical problem to be further solved by the present invention is to provide an adenovirus vector, a UTX adenovirus as well as a preparation method and an application thereof.

The technical problem to be further solved by the present invention is to provide a therapeutic application of UTX overexpression on model with hyperlipidemia and NAFLD.

Besides, we also constructed a UTX overexpression adenovirus vector and transduced it into mice through tail-vein injection to specifically overexpress UTX gene in liver. H&E staining and hepatic triglyceride detection result ensure the therapeutic action of UTX gene for hyperlipidemia and NAFLD. Further, we should put more efforts on establishing an application of a UTX gene in preparation of drugs for preventing or treating lipid diseases.

Technical solution: in order to solve the technical problems above, the technical solution of the present invention is as follows: the present invention comprises an application of a UTX gene in preparation of drugs for preventing or treating lipid diseases.

The present invention further comprises an application of a UTX gene in preparation of lipid-lowering drugs.

The present invention further comprises a method for knocking out a UTX gene from a mouse liver which specifically knocks out a UTX gene from a liver tissue by constructing a genetically engineered mouse, comprising the following steps of:

1) mating a female UTX^{f/f} mouse with a male albcre mouse to obtain a male UTX^{f/y}:albcre mouse through gene identification;

2) mating the male UTX^{f/y}:albcre mouse with the female UTX^{f/f} mouse to obtain a female UTX^{f/f}:albcre mouse or a male UTX^{f/y}:albcre mouse; and

3) genetically identifying the female UTX^{f/f}:albcre mouse to obtain a UTX knockout mouse.

The present invention further comprises an adenovirus vector, which comprises a UTX gene, and the vector can effectively overexpress the UTX gene in a liver cell.

The adenovirus vector is a pAdEasy-1 vector.

The present invention further comprises a UTX overexpression adenovirus, which comprises the adenovirus vector.

The present invention further comprises a preparation method of a UTX overexpression adenovirus, which comprises the following steps of:

1) obtaining a cDNA: extracting an RNA from a mouse liver tissue and reversely transcribing a cDNA;

2) obtaining a UTX gene: amplifying a UTX gene by PCR using a cDNA of the liver tissue as a template;

3) constructing a UTX adenovirus overexpression vector: cloning the UTX gene to a pShuttle vector, linearizing a restriction enzyme PmeI, co-transfecting the UTX gene with an adenovirus framework plasmid p AdEasy-1 vector into a BJ5183 strain, and screening a recombinant positive plasmid to obtain the UTX adenovirus overexpression vector; and

4) transfecting the UTX adenovirus overexpression vector into cultured AD-293 cells for continuously culturing for 7 days to 10 days, discarding a cell culture supernatant, collecting a cell suspension into an EP tube, repeatedly freezing/unfreezing in a methanol ice bath and a water bath, and shaking the cell briefly after unfreezing to obtain the pAdEasy-UTX adenoviruses.

A sequence of a forward primer amplified by PCR in the step 2) is shown in SEQ ID NO:1, and a reverse primer is shown in SEQ ID NO:2.

The cultured AD-293 cells in the step 4) are the AD-293 cells evenly inoculated in a culture dish at a density of 7 to 8*10⁵/ml in a 5% CO2 incubator under 37° C. until a cell fusion degree reaches 70% to 80%.

The present invention further comprises applications of the adenovirus vector and the UTX overexpression adenovirus in preparation of drugs for preventing or treating lipid diseases.

The present invention further illustrates a method for overexpressing UTX gene specifically in a mouse liver, comprising the following steps of:

1) To induce hyperlipidemia and non-alcoholic fatty acid liver disease (NAFLD), 6-week-old mice were fed with a high-fat diet (HFD, fat content 60%; Research Diets, New Brunswick, N.J.), called HFD mouse,

2) 2 months later, purified pAdEasy-UTX adenoviruses (1.5*10⁸ plaque-forming units per mouse) were transduced into mice through tail-vein injection to specifically upregulate UTX expression in liver.

3) Three days after the injection, all mice were killed and the livers were collected for further analysis lipid contents.

Beneficial effects: compared with the prior art, the present invention has the advantages as follows.

1) According to the present invention, the liver-specific UTX knockout mouse is obtained by hybridization of the UTX flox mouse and the Albcre mouse; and it is found for the first time that the contents of cholesterol and triglyceride in blood of the albcre mouse after knocking out the UTX are significantly increased, so that the application of UTX in preparation of lipid-lowering drugs is proposed.

2) According to the present invention, a UTX overexpression adenovirus is obtained for the first time, through which a UTX protein is over-expressed in the liver cell. The adenovirus vector contains a UTX nucleotide sequence, and after the UTX is over-expressed, lipid secretion of the liver cell can be inhibited, thus achieving the purpose of lowering the lipid.

3) According to the present invention, purified UTX overexpression adenovirus were transduced into mice through tail-vein injection to specifically upregulate UTX expression in liver. And three days later, all mice were killed and the livers were collected for further analysis to detect lipid-lowering function of UTX gene.

4) The present invention provides an available laboratory basis for preparing lipid-lowering drugs, so that the UTX can be used for preparing drugs affecting the lipid, and a new research method is provided for researching the occurrence and development of dyslipidemia.

### BRIEF DESCRIPTION OF THE DRAWINGS

FIG. 1A. illustrates PCR identification results of different UTX genotypes;

FIG. 1B, shows a knockout efficiency of a mRNA level detected by qRT-PCR;

FIG. 1C. shows a knockout efficiency of a protein level detected by Western blot;

FIG. 1D. shows quantitative analysis of Western blot results;

FIG. 2 illustrates detection result of an overexpression efficiency of UTX in a HepG2 liver cell; and

FIG. 3 illustrates a triglyceride detection result after the UTX is over-expressed in the HepG2 liver cell; and

FIG. 4A illustrates lipid contents in liver tissue sections after the UTX is over-expressed in the HFD mouse by H&E staining;

FIG. 4B illustrates a hepatic triglyceride detection result in the UTX over-expressed HFD mouse.

### DETAILED DESCRIPTION

### (I) Main Reagents

A UTX antibody used in the research is purchased from Genetex Company in America; a GAPDH antibody is purchased from Nanjing Bioworld Company; a Trizol reagent, a mRNA reverse transcription kit and a quantitative PCR kit are purchased from Invitrogen Company in America; a protein lysate, a protease inhibitor and protease K are purchased from Pierce Company in America; an ECL chemiluminescence detection kit is purchased from Nanjing Tiangen Biology Co., Ltd.; a PVDF membrane is purchased from Millipore Company in America; ordinary Taq enzyme is purchased from Nanjing Vazyme Company; DMEM medium, fetal bovine serum, pancreatin, PBS and streptomycin are purchased from Gibico Company in America; and glucose is purchased from Sigma Company in America.

### (II) Main Instruments

UTL ultra-low temperature freezer: Thermo Company, U.S.
Gradient PCR instrument: Eppendorf Company, France
Gel imaging system: Bio-rad, U.S.
Electrophoresis apparatus, electrophoresis tank: Bio-rad, U.S.
Life ViiA7 fluorescent quantitative PCR instrument: Life Company, U.S.

### Nanodrop2.0: Thermo Fisher Scientific, U.S.

Water bath tank: PolyScience Company, U.S.
Electronic balance: Shanghai Precision Instrument Co., Ltd.
Cell incubator: Sanyo Company, Japan

### Embodiment 1 Method for Knocking Out UTX Gene

### 1. Breeding of Mice

A C57BL/6J mouse, a UTX flox mouse and an Albcre tool mouse were all purchased from the Model Animal Research Center of Nanjing University. Experimental mice were raised in a SPF-level animal room in strict accordance with the requirements set forth in the Regulations for the Administration of Experimental Animals. The environment conformed to a biological rhythm of 12-hour diurnal cycle and the experimental mice were free to eat food and drink water. Breeding cages were configured according to a manner of one male mouse matched with two female mice, toes were numbered 7 days to 14 days after the mice were born, and tails were cut to identify a genotype. The male and female mice were raised separately in different cages 21 days after birth. Wild UTX^{f/f} mice of the same age and the same sex were used as control mice. 2. Genotype identification of mice

1) Extraction of Tail DNA

A. 1 cm to 2 cm of a tail tissue of a mouse or a mouse toe was cut;

B. 300 μl of lysate and 5 μl of protease K (with a concentration of 20 mg/ml) were added, and the mixture was kept at 55° C. overnight until the tissue was completely digested;

C. 600 μl of absolute ethyl alcohol was added and evenly mixed;

D. the mixture was centrifuged at 12,000 rpm for 2 min, and then a liquid was removed;

E. 600 μl of 70% ethanol was added for washing, and centrifuged at 12,000 rpm for 2 min, and then a liquid was removed;

F. the remaining was dried in air; and

G. 300 μl of sterilized distilled water was added to dissolve a DNA, and then the mixture was stored in a refrigerator at 4° C.

2) Genotype Identification of Mice by PCR

Using the extracted tail DNA as a template, the Taq enzyme reagent from Vazyme Company was added into 8-tube strips according to the following table, evenly mixed and centrifuged.

| Component | Concentration | Volume | |
|---|---|---|---|
| 2xReaction Mix | | 10 | μl |
| Forward primer | 10 μM | 0.5 | μl |
| Reverse primer | 10 μM | 0.5 | μl |
| DNA | | 2 | μl |
| ddH₂O | | 7 | μl |
| Total Volume | | 20 | μl |

The mixture was placed in a PE2400 PCR instrument for PCR amplification, and PCR cycle parameters were as follows:

| | | | | |
|---|---|---|---|---|
| 95° C. | 5 | min | | |
| 95° C. | 30 | sec | | |
| 56° C. | 30 | sec | {close oversize brace} | 36 cycles |
| 72° C. | 30 | sec | | |
| 72° C. | 7 | min | | |

Primer sequences were as follows:

| Primer | Forward primer or | |
|---|---|---|
| name | reverse primer | Primer sequence (5′-3′) |
| UTX | F (SEQ ID NO.: 3) | AAGGTTCTGCTGGCATAGTGGA |
| | R (SEQ ID NO.: 4) | CTACATACAGAAGCCATAGCCAGT |
| Cre712 | F (SEQ ID NO.: 5) | AATGCTTCTGTCCGTTTGC |
| | R (SEQ ID NO.: 6) | ACCAGAGTCATCCTTAGCG |

10 μl of PCR products and 1.0% to 2.0% agarose/ethidium bromide were taken for gel electrophoresis, and analysis was carried out by a UVP gel density scanner and an analysis software (UVP, Inc.) thereof; and target strip with a corresponding size could be seen positively.

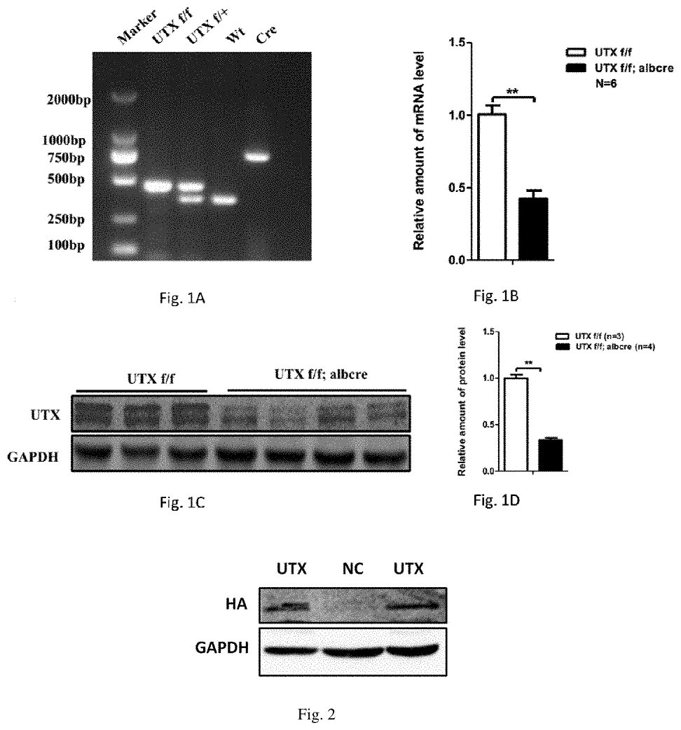

3. A female UTX^{f/f} mouse (purchased from Jackson Laboratory in U.S.) was mated with a male albcre mouse (purchased from Jackson Laboratory in U.S.) to obtain a male UTX^{f/y}:albcre mouse through gene identification by the method in the step 2; and the male mouse was further mated with the female UTX^{f/f} mouse to obtain a female UTX^{f/f}:albcre mouse or a male UTX^{f/y}:albcre mouse, and then a subsequent experiment was performed. In view that the UTX is an X sex chromosome linkage gene, and the female mouse contains two alleles, but the male mouse contains only one allele, so the female UTX^{f/f}:albcre knockout mouse was mainly used in the research herein. The mouse genotype was identified by PCR (see FIG. 1A).

A knockout efficiency was identified by qRT-PCR and Western blot respectively. 1) Identification of knockout efficiency of mouse by qRT-PCR: appropriate amounts of liver tissues of a UTX^{f/f}:albcre knockout mouse and a UTX^{f/f} mouse were selected respectively, and RNAs in the mouse liver tissue were extracted by a Trizol® reagent from Invitrogen Company.

An integrity of the RNA was detected by formaldehyde denaturing electrophoresis. After a total RNA concentration was detected by NanoDrop 2.0, reverse transcription was performed by a reverse transcription kit from Promega Company to obtain cDNAs. Further, a quantitative kit from Invitrogen Company was used for detection, and the results showed that an mRNA expression level of the UTX^{f/f}:albcre knockout mouse was lower than that of the control mouse, and the knockout efficiency could reach 60% to 70% (FIG. 1B).

2) Identification of knockout efficiency of mouse by Western blot: appropriate amounts of liver tissues of a UTX^{f/f}:albcre knockout mouse and a UTX^{f/f} mouse were selected respectively, a RIPA protein lysate and a protease inhibitor cocktail were used to collect liver tissue samples, total proteins were extracted respectively, a BCA kit from Pierce Company was used to determine protein concentrations, and then Western blot detection was performed. Results showed that a UTX mRNA expression level was consistent with a protein level, and the UTX knockout efficiency reached 70% (FIG. 1C-D), which suggested that a liver-specific UTX knockout mouse had been successfully constructed.

4. Serum Collection and Blood Biochemical Determination of Mouse

1) Preparation of mouse: the mouse was placed into a clean cage at 5:00 p.m. prior to the experiment and fasted for 16 h until 9:00 a.m. next day. During fasting, the mouse drunk water normally.

2) After the mouse was acclimatized for 30 min, about 500 μl of blood was collected by a capillary tube through an orbit of the mouse, the blood was placed in a 1.5 ml EP tube without an anticoagulant, and then the blood sample was placed in a refrigerator at 4° C. to coagulate for 2 h.

3) The coagulated blood sample was centrifuged at 4° C. for 15 min at a speed of 4000 g by a refrigerated centrifuge.

4) A supernatant (serum) was transferred to a new EP tube for a subsequent experiment. If needed, the serum was packaged according to an appropriate volume as required and stored in a refrigerator at −80° C. Repeated freezing and unfreezing were forbidden.

5) The blood sample was sent to the Laboratory Department of Nanjing Maternity and Child Health Care Hospital to detect relevant biochemical indexes.

Results of blood biochemical indexes of the UTX^{f/f}:albcre mouse were shown in Table 1. The UTX^{f/f}:albcre mouse had significantly higher blood cholesterol and triglyceride levels in a fasting state, but had no significant difference in a blood glucose level; wherein the cholesterol mainly comprised LDL-C and HDL-C, while the results showed that the LDL-C contents in the blood of the UTX^{f/f}:albcre mouse were increased significantly, but ratios of the HDL-C contents were decreased significantly after being corrected by a total cholesterol content (Chol) or the LDL-C although the HDL-C was also increased to some extent.

Table 1 Blood Biochemical Indexes of UTX^{f/f}:albcre Liver Knockout Mouse (Female Mouse)

| | UTX^{f/f} | UTX^{f/f}:albcre |
|---|---|---|
| Parameters | (n = 6) | (n = 8) |
| Chol | 2.37 +/− 0.41 | 4.32 +/− 0.95** |
| TG | 1.41 +/− 0.77 | 2.48 +/− 0.84* |
| LDL-C | 0.28 +/− 0.06 | 0.94 +/− 0.37** |
| HDL-C | 2.07 +/− 0.27 | 2.98 +/− 0.37** |
| HDL-C/TC | 0.883 +/− 0.062 | 0.706 +/− 0.084** |
| HDL-C/LDL-C | 7.474 +/− 0.993 | 3.628 +/− 1.387** |
| Glu | 3.87 +/− 0.35 | 3.80 +/− 0.28 |

Data are expressed as mean ± SD,
*p < 0.05,
**p < 0.001

### Embodiment 2 Preparation of UTX Overexpression Adenovirus

1) Extraction of RNA from liver tissue and reverse transcription of cDNA: an appropriate amount of liver tissues of a C57BL/6J mouse was selected, and an RNA was extracted from the mouse liver tissue using a miRNeasy® total RNA extraction kit from Qiagen Company. An integrity of the RNA was detected by formaldehyde denaturing electrophoresis, a total RNA concentration was detected by NanoDrop 2.0, and then reverse transcription was performed by a HiScript 1st Strand cDNA Synthesis Kit reverse transcription kit from Vazyme Company to obtain a template cDNA.

2) Amplification of UTX gene by PCR: PCR primers were designed according to a UTX mRNA sequence in NCBI GeneBank, wherein a forward primer (containing a BamHI/NotI enzyme cutting site and a HA tag sequence) was: TAAGGATCCACCATGGCTTACCCATACGATGTTCCAGATTACGCTTCGAAATCCTGCGGAGT GTCGC (SED ID NO. 1); and a reverse primer was TCGGCGGCCGCTTACT TTCTGAATAGCAGAAAAGGTC (SED ID NO. 2);; and PCR amplification was performed with Phantamax Super-Fidelity DNA Polymerase (Vazyme Company) by using a cDNA of the liver tissue as a template. Amplified products were subjected to PCR product purification, cloned and transformed into TOP10 competent cells, and coated on a solid medium containing LB, and further, positive bacteria were selected, and sent for sequencing after plasmid extraction. After successful sequencing, the UTX gene was cloned to a pShuttle vector (purchased from Shanghai Zeye Biotechnology), which was co-transfected with an adenovirus framework plasmid pAdEasy-1 vector (purchased from Shanghai Zeye Biotechnology) into a plasmid recombinant host strain BJ5183 after a restriction enzyme PmeI (purchased from NEB Company) was linearized. A recombinant positive plasmid was screened, named as vAD-UTX, and further amplified for adenovirus coating after sequencing identification.

3) UTX adenovirus coating: AD-293 cells (stored in the laboratory) were evenly inoculated in a 6 cm culture dish at a density of 7 to 8*10⁵/ml in a 5% CO₂ incubator under 37° C. When a cell fusion degree reached about 70%, the linearized vAD-UTX plasmid was transfected to the AD-293 cells, continuously cultured for 7 days to 10 days, then a cell culture supernatant was discarded, a cell suspension was collected into an EP tube, and repeatedly frozen/unfrozen in a methanol ice bath and a water bath at 37° C., and the cell was shaken briefly after unfreezing to obtain the adenovirus.

### Embodiment 3 Identification of Overexpression Efficiency of UTX Overexpression Adenovirus Infecting HepG2 Cells

1) Frozen HepG2 cells (stored in the laboratory) were thawed in a DMEM culture solution containing 10% fetal bovine serum, 50 μg/ml penicillin and 100 μg/ml streptomycin, and cultured in a 5% CO₂ incubator of constant temperature under 37° C. When the cells were in good condition and grown to a cell fusion degree of about 80%, the cells were digested with a digestive juice containing 0.01% EDTA and 0.25% trypsin, and passaged according to a ratio of 1 to 3.

2) When the cell fusion degree was about 50%, the UTX adenovirus obtained in the Embodiment 2 and a negative control adenovirus without the target gene UTX were respectively added for infection, and cultured for 48 h to 72 h, then cell samples were collected with a RIPA protein lysate and a protease inhibitor cocktail, total proteins were extracted, and protein concentrations were determined by BCA, and Western blot detection was performed.

The overexpression efficiency of the UTX in the HepG2 liver cell was detected. FIG. 2 showed the results of Western blot. Compared with a negative adenovirus control group, an adenovirus infection group had a higher content of exogenous HA-UTX protein (containing a tag protein HA), which indicated the successful preparation of the overexpression adenovirus.

### Embodiment 4 Detection of Triglyceride after UTX Adenovirus Infection of HepG2 Cells

1) HepG2 cells were cultured. When a fusion degree of the cells was about 50%, the UTX adenovirus obtained in the Embodiment 2 and a control adenovirus were respectively added for infection, cultured for 24 h, and then a culture medium was changed;

2) after culturing for 24 h, the culture medium was replaced by a serum-free DMEM culture medium for continuously culturing for 24 h to promote cell synchronization, so that most cells were in a Gₒ phase;

3) after continuously culturing for 24 h, the medium was replaced with a sugar-free DMEM culture medium containing 30 mmol/L glucose for treating the cells for 24 h; and

4) a cell supernatant was collected, and automatic biochemical analysis was performed to detect extracellular triglyceride content in the laboratory of Nanjing Maternity and Child Health Care Hospital.

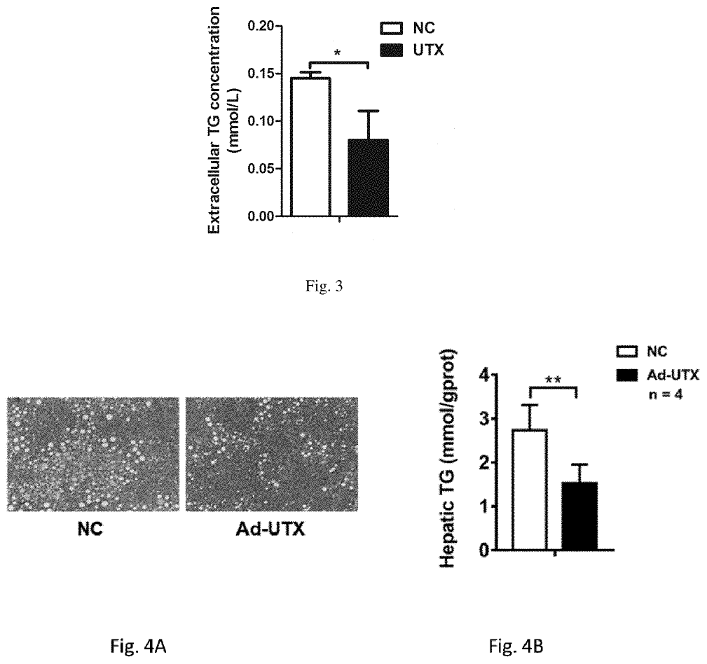

After the UTX was over-expressed in the HepG2 liver cells, glucose was used to stimulate for 24 h, and then the cell supernatant was collected for triglyceride detection. FIG. 3 showed the results. After the UTX was over-expressed, lipid secretion of the liver cells could be significantly decreased by about 50% in comparison with the control group.

### Embodiment 5 Phenotypes of UTX Overexpression Mice

1. Acquisition of UTX Overexpression Mouse

To induce hyperlipidemia and non-alcoholic fatty acid liver disease (NAFLD), 6-week-old mice were fed with a high-fat diet (HFD, fat content 60%; Research Diets, New Brunswick, N.J.). 2 months later, purified pAdEasy-UTX adenoviruses (1.5*10 plaque-forming units per mouse) were transduced into mice through tail-vein injection to specifically upregulate UTX expression in liver. Three days after the injection, all mice were killed and the livers were collected for further analysis.

2. Serum Collection and Blood Biochemical Determination of the UTX Overexpression Mouse 1) Methods of Serum Collection were Same as Shown in Embodiment 1-4.

2) Results of blood biochemical indexes of the UTX overexpression mouse were shown in Table 2. The UTX overexpression mouse had significantly lower blood cholesterol and triglyceride levels, but had no significant difference in a blood glucose level; wherein the cholesterol mainly comprised LDL-C and HDL-C, while the results showed that contents in the UTX overexpression mouse were decreased to 35% and increased 1.5 fold, respectively. And ratios of the HDL-C contents were increased significantly after being corrected by a total cholesterol content (Chol) or the LDL-C, in accordance with the HDL-C was increased to some extent.

TABLE 2
Blood Biochemical Indexes of UTX Liver
Overexpression Mouse (Female Mouse)

| Parameters | NC | Ad-UTX |
|---|---|---|
| Chol | 6.52 +/− 1.02 | 4.28 +/− 0.89** |
| TG | 1.83 +/− 0.33 | 1.25 +/− 0.26* |
| LDL-C | 0.64 +/− 0.15 | 0.24 +/− 0.08** |
| HDL-C | 2.07 +/− 0.48 | 2.96 +/− 0.52* |
| HDL-C/TC | 0.62 +/− 0.04 | 0.76 +/− 0.06** |
| HDL-C/LDL-C | 4.13 +/− 0.82 | 7.63 +/− 1.19** |
| Glu | 4.83 +/− 0.43 | 4.92 +/− 0.39 |

Data are expressed as mean ± SD, n = 4,
*p < 0.05,
**p < 0.001

### Embodiment 6 Function of UTX Overexpression on Hepatic Lipid Content

1. H&E Staining Once UTX overexpression mouse were executed, liver tissues were harvested. A small piece of liver was cut and submerged in 4% paraformaldehyde. The sample was sent to Service Biology (Wuhan, Hubei, China) for hematoxylin and eosin (H&E) staining to detect lipid content.

2. Hepatic Triglyceride Content

Once UTX overexpression mouse were executed, liver tissues were harvested, frozen immediately in liquid nitrogen, and stored at −80° C. for subsequent analysis. A small piece of liver were homogenerated in PBS by the ratio of 1:9 (g:ml), and cell supernatant was collected. Automatic biochemical analysis was performed to detect extracellular triglyceride content in the laboratory of Nanjing Maternity and Child Health Care Hospital.

After the UTX was over-expressed in hyperlidemia and NAFLD mice, FIG. 4 showed the lipid content by H&E staining and biochemical analysis. After the UTX was over-expressed, H&E staining showed that number and size of lipid dropet in liver tissues were decreased significantly (FIG. 4A). And the hepatic lipid cintent could be significantly decreased by about 50% in comparison with the control group (FIG. 4B).

Various embodiments of the invention have been described above, and the description above is exemplary, but not exhaustive, and the invention is not limited to the disclosed embodiments. Many modifications and variations are apparent to those of ordinary skills in the art without departing from the scope and spirit of the described embodiments.

What is claimed is:

1. A method for knocking out a UTX gene from a mouse liver which specifically knocks out a UTX gene from a liver tissue by constructing a genetically engineered mouse, comprising the following steps of:
1) mating a female UTX^{f/f} mouse with a male albcre mouse to obtain a male UTX^{f/y}:albcre mouse through gene identification;
2) mating the male UTX^{f/y}:albcre mouse with the female UTX^{f/f} mouse to obtain a female UTX^{f/f}:albcre mouse or a male UTX^{f/f}:albcre mouse; and
3) genetically identifying the female UTX^{f/f}:albcre mouse to obtain a liver-specific UTX knockout mouse.

2. A preparation method of a UTX overexpression adenovirus, comprising the following steps of:
1) obtaining a cDNA: extracting an RNA from a mouse liver tissue and reversely transcribing a cDNA;
2) obtaining a UTX gene: amplifying a UTX gene by PCR using a cDNA of the liver tissue as a template;
3) constructing a UTX adenovirus overexpression vector: cloning the UTX gene to a pShuttle vector, linearizing a restriction enzyme PmeI, co-transfecting the UTX gene with a pAdEasy-1 vector into a BJ5183 strain, and screening a recombinant positive plasmid to obtain the UTX adenovirus overexpression vector; and
4) transfecting the UTX adenovirus overexpression vector into cultured AD-293 cells for continuously culturing for 7 days to 10 days, discarding a cell culture supernatant, collecting a cell suspension into an EP tube, repeatedly freezing/unfreezing in a methanol ice bath and a water bath, and shaking the cell briefly after unfreezing to obtain the adenovirus.

3. The preparation method of the UTX overexpression adenovirus according to claim 2, wherein a sequence of a forward primer amplified by PCR in the step 2) is shown in SEQ ID NO:1, and a reverse primer is shown in SEQ ID NO:2.

4. The preparation method of the UTX overexpression adenovirus according to claim 2, wherein the cultured AD-293 cells in the step 4) are the AD-293 cells evenly inoculated in a culture dish at a density of 7 to 8*10⁵/ml in a 5% CO2 incubator under 37° C. until a cell fusion degree reaches 70% to 80%.

5. The preparation method of the UTX overexpression adenovirus according to claim 2, wherein the UTX overexpression adenovirus is in preparation of drugs for preventing or treating lipid diseases.

6. A process for treating of preventing a lipid disease by administering a drug that increases an activity of UTX on a patient in need of lipid disease treatment or prevention.

7. The process according to claim 6, wherein the drug is one or more selecting from a group consisting of a UTX overexpression adenovirus, an exogenous UTX protein and a chemical compound that upregulates expression or activity of UTX.


SEQUENCE LISTING (6 sequences)

SEQ ID NO: 1 - DNA, 67 bp, Artificial Sequence
  It is synthesized.
taaggatccaccatggcttacccatacgatgttccagattacgcttcgaaatcctgcggagtgtcgc

SEQ ID NO: 2 - DNA, 37 bp, Artificial Sequence
  It is synthesized
tcggcggccgcttactttctgaatagcagaaaaggtc

SEQ ID NO: 3 - DNA, 22 bp, Artificial Sequence
  It is synthesized.
aaggttctgctggcatagtgga

SEQ ID NO: 4 - DNA, 24 bp, Artificial Sequence
  It is synthesized.
ctacatacagaagccatagccagt

SEQ ID NO: 5 - DNA, 19 bp, Artificial Sequence
  It is synthesized.
aatgcttctgtccgtttgc

SEQ ID NO: 6 - DNA, 19 bp, Artificial Sequence
  It is synthesized.
accagagtcatccttagcg